(19) United States
(12) Patent Application Publication
(10) Pub. No.: US 2024/0225444 A1
(43) Pub. Date: Jul. 11, 2024

(54) WIRELESS MULTI-LEAD ELECTROCARDIOGRAM SYSTEM

(71) Applicant: Rany M. Saleh, Jersey City, NJ (US)

(72) Inventor: Rany M. Saleh, Jersey City, NJ (US)

(21) Appl. No.: 18/406,410

(22) Filed: Jan. 8, 2024

Related U.S. Application Data
(60) Provisional application No. 63/437,900, filed on Jan. 9, 2023.

(51) Int. Cl. A61B 5/00; A61B 5/332; A61B 5/339

(52) U.S. Cl. CPC A61B 5/0006; A61B 5/332; A61B 5/339; A61B 2560/0456; A61B 2562/08

(57) ABSTRACT

A wireless electrocardiogram (ECG) system is provided. The wireless ECG system includes a plurality of wireless contact leads positioned on different areas of a body of a patient. Each of the wireless contact leads wirelessly send is a plurality of signals received from the body of the patient. An processing unit receives the signals from the wireless contact leads. The processing unit processes the signals into data indicative of electrical activity of the body. A charging component is coupled to the processing unit. The charging component includes a plurality of slots for charging each of the wireless contact leads.

### RELATED APPLICATIONS

This application claims priority to U.S. provisional application No. 63/437,900 filed on Jan. 9, 2023, the content of which is included herein in its entirety.

### BACKGROUND

Typically, a standard electrocardiogram (ECG) machine records the electrical signals in the heart. This process is painless and is used to detect heart problems and monitor the heart's health quickly. ECGs are often performed in a health care provider's office, a clinic, or a hospital room. ECG machines are used to perform ECG testing. Moreover, ECG machines have become a norm in hospital rooms, operating rooms, stress testing labs, cardiac labs, ambulances, and even urgent care centers.

The standard ECG machine is commonly referred to as a 12-lead ECG, which includes ten different wired leads connected to the patient for detecting electric vectors-signals in the heart. These wired contact leads present many problems, including for example determining which wires go with which leads, resulting in inaccurate lead placement and ECG results. In addition, standard ECG machines are bulky with leads that are intertwined requiring unravel and connect, resulting in significant time to perform testing and obtain results. In many operating rooms, cardiac labs, and radiology suites, ECG wires often interfere with procedures and slow the operator and staff.

### SUMMARY

The present disclosure describes a wireless ECG system for providing reliable ECG results. The advantages provided by the wireless ECG system include faster placement and removal of ECG leads because there are no wires involved. The wireless ECG system includes wireless ECG leads configured to allow for easier identification of the wireless ECG leads by altering their respective tops and bases. Also, the wireless ECG system eliminates the problems presented by overlying and overlapping wires. Moreover, one can use the wireless ECG system in hospitals and doctor offices. If a patient is at home or using telemedicine, the wireless ECG system may be used for ECG testing at home.

According to one aspect of the subject matter described in this disclosure, a wireless electrocardiogram (ECG) system is provided. The wireless ECG system includes a plurality of wireless contact leads for positioning on different areas of a body of a patient. Each of the wireless contact leads wirelessly send a plurality of signals received from the body of the patient. An ECG module receives the signals from the wireless contact leads. The ECG module processes the signals into data indicative of electrical activity of the body. A charging component is coupled to the ECG module. The charging component includes a plurality of slots for charging each of the wireless contact leads.

According to another aspect of the subject matter described in this disclosure, a charging system is provided. The charging system includes a plurality of wireless contact leads configured to receive signals from a body to perform a multi-lead electrocardiogram (ECG). A charging component includes a plurality of receivable slots for charging each of the wireless contact leads. Each of the receivable slots assigned to a particular wireless contact lead from the plurality of wireless contact leads. The charging component has a plurality of indicators to indicate the status of each of the wireless contact leads positioned in their respective receivable slots.

According to another aspect of the subject matter described in this disclosure, a method for performing operations of a wireless electrocardiogram (ECG) system is provided. The method includes providing a plurality of wireless contact leads positioned on different areas of a body of a patient. The method also includes sending, by each of the wireless contact leads, a plurality of signals received from the body. The signals from the wireless contact leads are received using a processing unit. The method further includes processing, using the processing unit, the signals into data indicative of electrical activity of the body. The method further includes charging, using a charging component, each of the wireless contact leads. The charging component includes a plurality of slots for charging each of the wireless contact leads.

According to another aspect of the subject matter described in this disclosure, an electrocardiogram (ECG) system is provided. The ECG system includes a plurality of wireless contact leads. A processing unit is configured to receive signals from the wireless contact leads and process the signals into data indicative of electrical activity of a body of a user.

According to another aspect of the subject matter described in this disclosure, a wireless contact lead is provided. The wireless contact lead includes a top portion configured to have markings indicative of a location of the wireless contact lead on a body. A base is coupled to the top portion configured to be positioned in a receivable slot of a charging system for charging the wireless contact lead.

Additional features and advantages of the present disclosure are described in, and will be apparent from, the detailed description of this disclosure.

### BRIEF DESCRIPTION OF THE DRAWINGS

The disclosure is illustrated by way of example, and not by way of limitation, in the figures of the accompanying drawings in which like reference numerals are used to refer to similar elements. It is emphasized that various features may not be drawn to scale and the dimensions of various features may be arbitrarily increased or reduced for clarity of discussion.

FIG. 1 is a schematic diagram of an arrangement of wireless contact leads on a body, in accordance with some embodiments.

FIG. 2 is a schematic diagram of a wireless electrocardiogram (ECG) system, in accordance with some embodiments.

FIG. 3 is a schematic diagram of a wireless ECG system implemented using a computer system, in accordance with some embodiments.

FIG. 4 is a schematic diagram of a computer system arranged to perform function associated with a wireless ECG system, in accordance with some embodiments.

FIG. 5 is a schematic diagram of a wireless contact lead when attached to electrodes, in accordance with some embodiments.

FIG. 6 is a schematic diagram of a charging component, in accordance with some embodiments.

FIG. 7 is a schematic diagram of wireless contact leads, in accordance with some embodiments.

FIG. 8 is a process flowgraph of operations performed by a wireless ECG system, in accordance with some embodiments.

### DETAILED DESCRIPTION

The figures and descriptions provided herein may have been simplified to illustrate aspects that are relevant for a clear understanding of the herein described devices, systems, and methods, while eliminating, for the purpose of clarity, other aspects that may be found in typical similar devices, systems, and methods. Those of ordinary skill may recognize that other elements and/or operations may be desirable and/or necessary to implement the devices, systems, and methods described herein. But because such elements and operations are well known in the art, and because they do not facilitate a better understanding of the present disclosure, a discussion of such elements and operations may not be provided herein. However, the present disclosure is deemed to inherently include all such elements, variations, and modifications to the described aspects that would be known to those of ordinary skill in the art.

The terminology used herein is for the purpose of describing particular example embodiments only and is not intended to be limiting. For example, as used herein, the singular forms “a”, “an” and “the” include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc., may be used herein to describe various elements, components, regions, layers and/or sections, these elements, components, regions, layers and/or sections should not be limited by these terms. These terms may be only used to distinguish one element, component, region, layer or section from another element, component, region, layer or section. That is, terms such as “first,” “second,” and other numerical terms, when used herein, do not imply a sequence or order unless clearly indicated by the context.

Described herein are example implementations of a wireless multi-lead electrocardiogram (ECG) system. The ECG system utilizes a wireless approach for collecting a patient's electrical signals from their body. In some embodiments, the wireless ECG system includes at least one wireless contact lead that may be positioned on various locations of a body to perform ECG testing. The wireless contact leads are configured to receive electrical vectors/signals from the body and sends these signals wirelessly to an ECG module. The ECG module processes these signals to be presented to a computer system. The computer system may perform further analysis or display these signals.

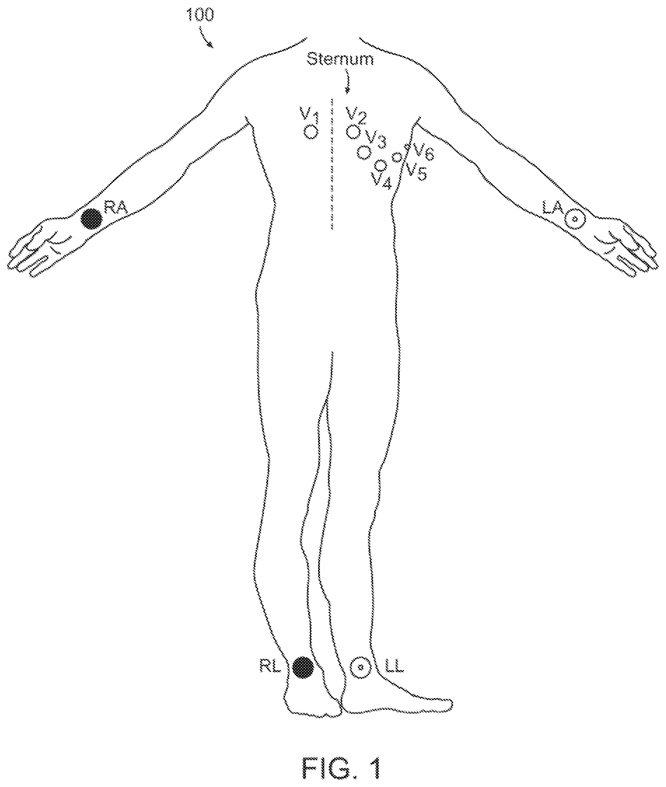

FIG. 1 is a schematic diagram of an arrangement of wireless contact leads on a body 100 of a patient. This embodiment shows a 12-lead ECG with ten wireless contact leads positioned throughout body 100. At least two of the ten wireless contact leads may be used. The ten wireless contact leads are as follows: right arm contact lead (RA), right leg contact lead (RL), left arm contact lead (LA), left leg contact lead (LL), and precordial contact leads V1-V6. The precordial contact leads V1-V6 are positioned around the sternum of body 100. Each contact lead RA, RL, LA, LL, and V1-V6 has electrical components stored within to communicate wirelessly to an ECG system using Bluetooth. In some embodiments, one may use other wireless communication systems similar to Bluetooth communication.

In some embodiments, one may use fewer wireless contact leads than those shown in FIG. 1 to perform other tests on a patient.

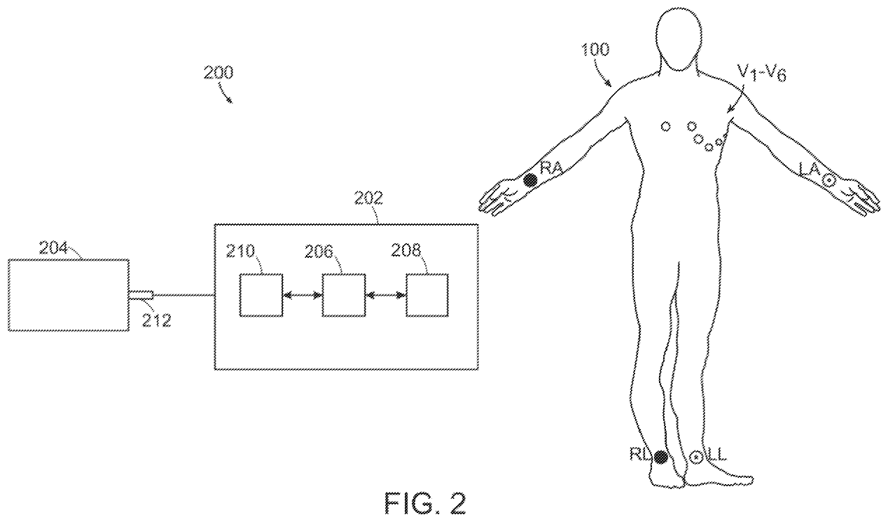

FIG. 2 is a schematic diagram of a wireless ECG system 200, in accordance with some embodiments. The wireless ECG system 200 includes wireless ECG module 202 and computer system 204. The wireless ECG module 202 is a standalone ECG unit that is configured to receive wireless signals from each contact lead RA, RL, LA, LL, and V1-V6 positioned on body 100. In particular, ECG module 202 includes a host processor 206, wireless module 208, and digital signal processor (DSP) 210. The wireless module 208 manages the data packets associated with sending and receiving information between ECG module 202 and contact leads RA, RL, LA, LL, and V1-V6. The wireless module 208 may use wireless communication protocols, such as Bluetooth and the like. A digital signal processor (DSP) 210 may be used to process the wireless signals received from contact leads RA, RL, LA, LL, and V1-V6 to retrieve from each leads RA, RL, LA, LL, and V1-V6 their corresponding electrical signals from body 100.

In some implementations, wireless module 208 may utilize the Bluetooth Classic standards for processing wireless signals. In this instance, wireless module 208 operates in a 2.4 GHz ISM band (2.402 GHz-2.480 GHz). Wireless module 208 includes 79 channels with 2 MHz spacing. Each contact lead RA, RL, LA, LL, and V1-V6 may be assigned at least one of the channels. Wireless module 208 may have a transmit power of less than or equal to 100 mW or 20 dBm and a receive power of less than or equal to −70 dBm for each contact lead RA, RL, LA, LL, and V1-V6.

In some implementations, wireless module 208 may utilize the Bluetooth LE (low energy) standards for processing LE 1M PHY, LE 2M PHY, and other LE-coded wireless signals. In this instance, wireless module 208 operates in a 2.4 GHz ISM band (2.402 GHz-2.480 GHz). Wireless module 208 includes 79 channels with 1 MHz spacing. Each contact lead RA, RL, LA, LIL, and V1-V6 may be assigned at least one of the channels. Wireless module 208 may have a transmit power of less than or equal to 100 mW or 20 dBm to communicate with each contact lead RA, RL, LA, LL, and V1-V6. Moreover, wireless module 208 includes a receive power of less or equal to −70 dBm for both LE 2M PHY and LE 1M PHY wireless signals for each contact lead RA, RL, LA, LL, and V1-V6. For LE-coded wireless signals, the maximum receive power may be −75 dBm or −82 dBm for each contact lead RA, RL, LA, LL, and V1-V6, depending on the data rate of the wireless signal.

Other standards besides those described herein may be used by wireless module 208.

The host processor 206 is provided to control the operations of wireless module 208 and DSP 210. Each lead contact RA, RL, LA, LL, and V1-V6 may be assigned a distinct channel in ECG module 202. This minimizes errors and/or corruption of the data received at ECG module 202. Moreover, one may analyze the wireless signals for each contact lead RA, RL, LA, LL, and V1-V6 individually using DSP 210. In some embodiments, host processor 206 may designate the wireless signals for each contact lead RA, RL, LA, LL, and V1-V6 for storage in a dedicated memory local to ECG module 202, or remotely for later processing.

Once the wireless signals from each lead contact RA, RL, LA, LL, and V1-V6 are processed, this data may be sent to computer system 204 for visualization. This data is indicative of the electrical vectors/signals of the heart, as an example. Moreover, the data may be displayed by computer system 204, or a 12-lead ECG printout may be generated by computer system 204. In these embodiments, a wireless connection may send the data associated with the electrical vectors/signals of the heart between ECG module 202 and computer system 204. In these embodiments, a wired connection through a USB port 212, or other input port, sends this data between ECG module 202 and computer system 204.

In some embodiments, the data may be indicative of the electrical vectors/signals of the body. In one example, the data may be indicative of the electrical vectors/signals of the brain. In one example, the data may be indicative of the electrical vectors/signals of the leg. The data may be indicative of other parts of the body besides those described. In this case, ECG module 202 may be configured to process signals received from other parts of the body besides that of the heart, such as the head, back, and the like.

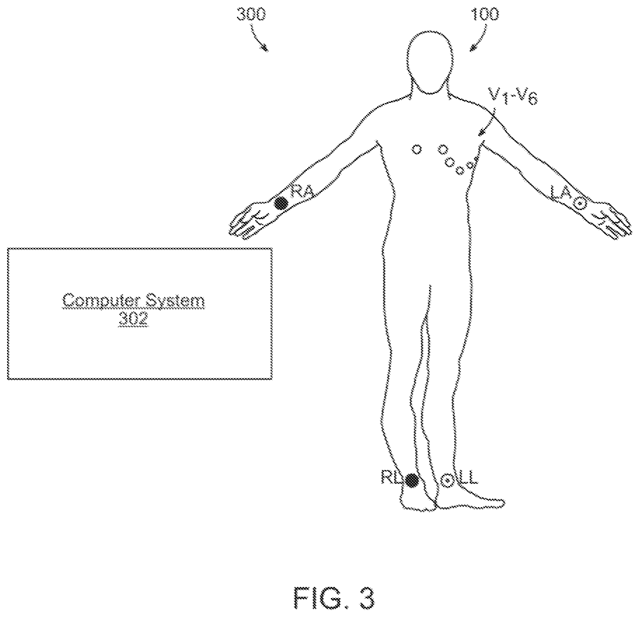

FIG. 3 is a schematic diagram of a wireless ECG system 300 implemented using a computer system 302. In this embodiment, ECG module 202 of FIG. 2 is replaced with a computer system 302, which may include software and hardware components that implement the operations of host processor 206, wireless module 208, and DSP 210.

Computer system 302 is configured to receive wireless signals from each contact lead RA, RL, LA, LL, and V1-V6 positioned on body 100. In particular, computer system 302 utilizes the Bluetooth communication protocol to manage the data packets associated with sending and receiving information between computer system 302 and contact leads RA, RL, LA, LL, and V1-V6. Also, computer system 202 may be used to process the wireless signals received from contact leads RA, RL, LA, LL, and V1-V6 to retrieve from each lead RA, RL, LA, LL, and V1-V6 their corresponding electrical signals from body 100.

Each lead contact RA, RL, LA, LL, and V1-V6 may be assigned a distinct channel in computer system 302. This minimizes errors and/or corruption of the data received at computer system 302. Moreover, computer system 302 allows one to individually analyze the wireless signals for each contact lead RA. RL, LA, LL, and V1-V6. In some embodiments, computer system 302 may designate the wireless signals for each contact lead RA, RL, LA, LL, and V1-V6 for storage in a dedicated memory local to computer system 302, or remotely for later processing.

Once the wireless signals from each lead contact RA, RL, LA, LL, and V1-V6 are processed, computer system 302 may display the data. In one example, computer system 302 may generate a 12-lead ECG printout from the data because the data is indicative of the electrical vectors/signals of the heart. In another example, computer system 302 may generate a layout of the brain's activity because the data is indicative of the electrical vectors/signals of the brain. Computer system 302 may display the data indicative of other regions/parts of the body besides those described.

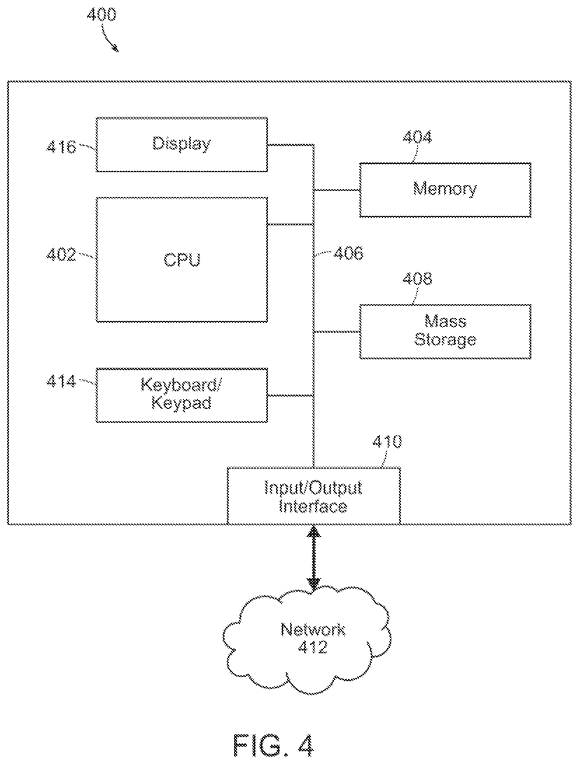

FIG. 4 shows a diagram of a computer system 400 arranged to perform functions associated with a wireless ECG system, including functions associated with computer system 302 of FIG. 3. The computer system 400 may be implemented as a virtual machine or a physical machine. The computer system 400 includes a central processing unit (CPU) 402, a memory 404, and an interconnect bus 406. The CPU 402 may include a single microprocessor or a plurality of microprocessors or special purpose processors for configuring computer system 400 as a multi-processor system. The memory 404 illustratively includes a main memory and a read only memory. The computer 400 also includes mass storage device 408 having, for example, various disk drives, tape drives, etc. Memory 404 also includes dynamic random-access memory (DRAM) and high-speed cache memory. In operation, memory 404 stores at least portions of instructions and data for execution by the CPU 402. Memory 404 may also contain computing elements, such as Deep In-Memory Architectures (DIMA), wherein data is sent to memory and a function of the data (e.g., matrix vector multiplication) is read out by CPU 402.

Mass storage 408 may include one or more magnetic disks, optical disk drives, and/or solid-state memories, for storing data and instructions for use by the CPU 402. At least one component of mass storage system 408, which may be in the form of a non-volatile disk drive, solid state, or tape drive, stores a database used for processing data and controlling functions associated with receiving user inputs and/or display data associated with a wireless ECG system, such as system 300. The mass storage system 408 may also include one or more drives for various portable media, such as a floppy disk, flash drive, a compact disc read only memory (CD-ROM, DVD, CD-RW, and variants), memory stick, or an integrated circuit non-volatile memory adapter (e.g., PC-MCIA adapter) to input and output data and code to and from computer system 400.

Computer system 400 may also include one or more input/output interfaces for communications, shown by way of example, as interface 410 and/or a transceiver for data communications via a network 412. Moreover, interface 410 may include communication protocols, such as Bluetooth and the like, to communicate to wireless devices. The wireless devices may include the wireless contact leads described herein. Data interface 410 may be a modem, an Ethernet card or any other suitable data communications device. To provide the functions of a processor as illustrated in FIG. 3, data interface 410 may provide a relatively high-speed link to network 412, such as an intranet, Internet, or the Internet, either directly or through another external interface. The communication link to network 412 may be, for example, optical, wired, or wireless (e.g., via satellite or cellular network). Computer system 400 may also connect via data interface 410 and network 412 to at least one other computer system to perform wireless ECG operations. Alternatively, computer system 400 may include a mainframe or other type of host computer system capable of Web-based communications via network 412. Computer system 400 may include software for operating a network application such as a web server and/or web client.

Computer system 400 may also include suitable input/output ports, that may interface with a portable data storage device, or use interconnect bus 406 for interconnection with a local display 416, computer mouse, and keyboard 414, and the like serving as a local user interface for programming and/or data retrieval purposes. A mouse may enable a user to position a pointer over a selectable icon and/or button on display 416 to enable the user to make selections and/or configure an object in display 416. Display 416 may include a touch screen capability to enable users to interface with system 400 by touching portions of the surface of display 416. Server operations personnel may interact with system 400 for controlling and/or programming the system from remote terminal devices via network 412.

The computer system 400 may run a variety of application programs and store associated data in a database of mass storage system 408. One or more such applications may include the operations of wireless ECG system 300 according to FIG. 3. The components contained in the computer system 400 may enable the computer system to be used as a server, workstation, personal computer, network terminal, mobile computing device, mobile telephone, System on a Chip (SoC), and the like. As disclosed above, the computer system 400 may include one or more applications, such as system 302. The system 400 may include software and/or hardware that implements a web server application. The web server application may include software such as HTML, XML, WML, SGML, PHP (Hypertext Preprocessor), CGI, and like languages.

The foregoing features of the disclosure may be realized as a software component operating in the system 400 where the system 400 includes Unix workstation, a Windows workstation, a LINUX workstation, or other type of workstation. Representative examples of other operation systems may be employed include Windows, MAC OS, and LINUX. In some embodiments, the software can optionally be implemented as a C language computer program, or a computer program written in any high level language including, for example, MATALB Javascript, Java, CSS, Python, Keras, Tensorflow, PHP, Ruby, C++, C, Shell, C#. Objective-C, Go, R, TeX, VimL, Perl, Scala, CoffeeScript, Emacs Lisp. Swift, Fortran, Visual BASIC, HDL, VHDL, and/or one or more versions of Verilog. Certain script-based programs may be employed such as XML, WML, PHP, and so on. The system 400 may use a digital signal processor (DSP).

As disclosed above, the mass storage 408 may include a database. The database may be any suitable database system, including the commercially available or open-source products, such as Microsoft Access, Sybase, SQL Server, MongoDB, SqlLite. The database can be implemented as a local or distributed database system. The database may be supported by any suitable persistent data memory, such as a hard disk drive, RAID system, tape drive system, floppy diskette, or any other suitable system. The system 400 may include a database that is integrated with the computer system 302, however, it will be understood that, in other implementations, the database and mass storage 408 can be an external element. The database may include information associated with wireless contact leads, client information, medical information, and the like.

In certain implementations, the system 400 may include an Internet browser program and/or be configured to operate as a web server. In some configurations, the client and/or web server may be configured to recognize and interpret various network protocols that may be used by a client or server program. Commonly used protocols include Hypertext Transfer Protocol (HTTP), File Transfer Protocol (FTP), Telnet, and Secure Sockets Layer (SSL), and Transport Layer Security (TLS), for example. However, new protocols and revisions of existing protocols may be frequently introduced. Thus, in order to support a new or revised protocol, a new revision of the server and/or client application may be continuously developed and released.

In one implementation, the system 400 includes a networked-based, e.g., Internet-based, application that may be configured and run on any combination of the other components of system 400. The computer system 400 may include a web server running a Web 2.0 application and the like. Web applications running on system 100 may use server-side dynamic content generation mechanisms, representative examples of which include Java servlets, CGI, PHP, and ASP. In certain embodiments, mashed content may be generated by a web browser running, for example, client-side scripting including JavaScript and/or applets on a wireless device.

In certain implementations, system 400 may include applications that employ Verilog HDL, VHDL, asynchronous JavaScript+XML (Ajax) and like technologies that use asynchronous loading and content presentation techniques. These techniques may include, for example, XHTML and CSS for style presentation, document object model (DOM) API exposed by a web browser, asynchronous data exchange of XML data, and web browser side scripting, e.g., JavaScript. Certain web-based applications and services may utilize web protocols including, for example, the services-orientated access protocol (SOAP) and representational state transfer (REST). REST may utilize HTTP with XML.

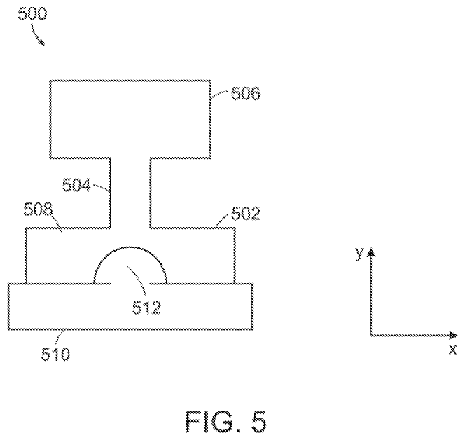

FIG. 5 is a schematic diagram of a wireless contact lead 500, in accordance with some embodiments. The wireless contact lead 500 is similar to contact leads RA, RL, LA, LL, and V1-V6 illustrated in FIGS. 1 and 2. In this embodiment, wireless contact lead 500 includes a base 502, a middle portion 504, and a top portion 506. Base 506 extends horizontally (parallel in the x-axis direction) with an indented portion 508 configured to receive an electrode sticker 510. The electrode sticker 510 includes a raised portion 512 that is received by indented portion 508, fastening the electrode sticker to base 506.

Base 502 may be of any shape. In some embodiments, the base of several wireless contact leads may have different shapes to distinguish between the wireless contact leads, and further reduce errors.

Middle portion 504 of wireless contact lead 500 extends vertically (parallel in the y-axis direction) and is positioned between base 502 and top portion 506. Moreover, middle portion 504 may include an antenna for communicating the electrical signals it receives from a body. In this embodiment, wireless contact lead 500 utilizes Bluetooth to communicate wirelessly. In some embodiments, wireless contact lead 500 may utilize other wireless communication standards for communicating wirelessly.

The top portion 506 of wireless contact lead 500 extends horizontally (parallel in the x-axis direction) and is connected to one of the distal ends of middle portion 504. The top portion 506 may be configured to include a marking indicating where on body 100 wireless contact lead 500 is to be positioned. For example, a marking of “RA” may be used for a wireless contact lead positioned on the right arm of a patient. In this case, one may use the references for contact leads RA, RL LA, LL, and V1-V6 as markings.

Top portion 506 may be of any shape. In some embodiments, the top portions of several wireless contact leads may have different shapes to distinguish between the other individual wireless contact leads. In some embodiments, the top portions of several wireless contact leads may be assigned different color arrangements to distinguish between the wireless contact leads.

When not connected to electrode sticker 510, base portion 502 may positioned in a powering station for charging wireless contact lead 500. The powering station may include a slot configured for securely receiving base 502. The base 502 is positioned within the slot for charging. This will be discussed further below.

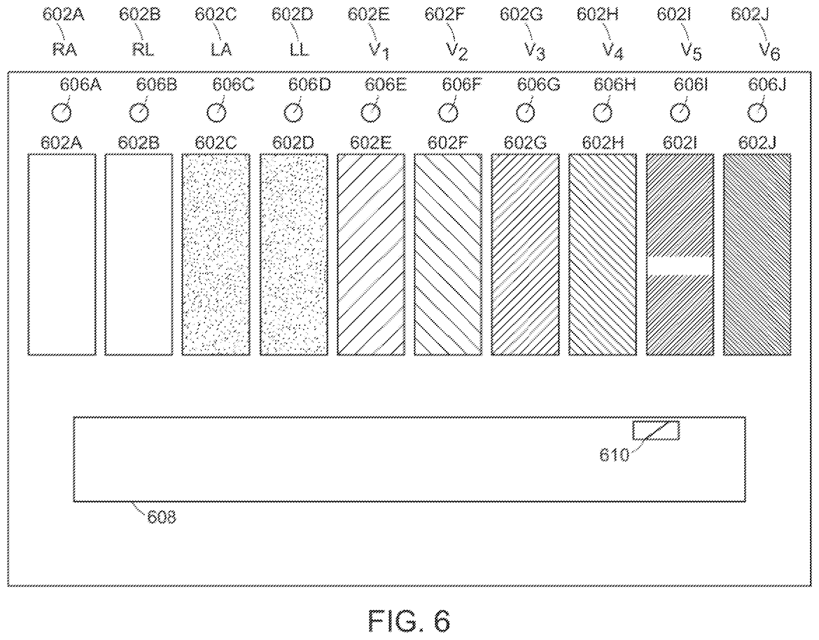

FIG. 6 is a schematic diagram of a charging component 600, in accordance with some embodiments. In particular, ECG module 202 may include a charging component 600 having a number of receivable slots 602A-602J for receiving contact leads RA, RL, LA, LL, and V1-V6, respectively. Each receivable slot 602A-602J may be labeled to a corresponding contact lead RA, RL, LA, LL, and V1-V6. When a contact lead RA, RL, LA, LL, and V1-V6 is properly positioned in its corresponding receivable slot 602A-602J, it is charged accordingly. The host processor 202 includes the appropriate protocols for charging each contact lead RA, RL, LA, LL, and V1-V6, including regulating the power needed to fully charge each contact lead RA, RL, LA, LL, and V1-V6. Each receivable slot 602A-602J may be assigned different colors and/or shadings corresponding to a contact lead RA, RL, LA. LL, and V1-V6.

In some embodiments, the charging component 600 may include an internal battery for storing power for charging. In some embodiments, the charging component 600 may be connected to a power source, such as a wall outlet, USB port, battery, and the like, for charging. In some embodiments, receivable slots 602A-602J may wirelessly charge each contact lead RA, RL, LA, LL, and V1-V6.

Moreover, charging component 600 includes labels 604A-604J associated with each contact lead RA, RL, LA, LL, and V1-V6. Charging indicators 606A-606J are provided for each contact lead RA, RL, LA, LL, and V1-V6. The charging indicators 606A-606J may be positioned above each receivable slot 602A-602J to indicate which of the contact leads RA, RL, LA, LL, and V1-V6 are currently being charged. A screen 608 may be positioned below receivable slots 602A-602J. Screen 608 may indicate which contact lead RA, RL, LA, LL, and V1-V6 has a poor connection or is misplaced. Also, screen 608 may show the overall battery level 610 of charging component 600 for charging contact leads RA, RL. LA, LL, and V1-V6.

In some embodiments, when contact leads RA, RL, LA, LL, and V1-V6 are correctly positioned in their corresponding receivable slot 602A-602J, the corresponding charging indicator 606A-606J flashes a light signal of a specific color a select number of times. Otherwise, a different color light signal may be flashed when any one or more of contact leads RA, RL, LA, LL, and V1-V6 are not correctly positioned in its corresponding receivable slot 602A-602J.

In some embodiments, the charging component 600 is integrally coupled to ECG module 202. In some embodiments, the charging component 600 is a stand-alone component separate from ECG module 202.

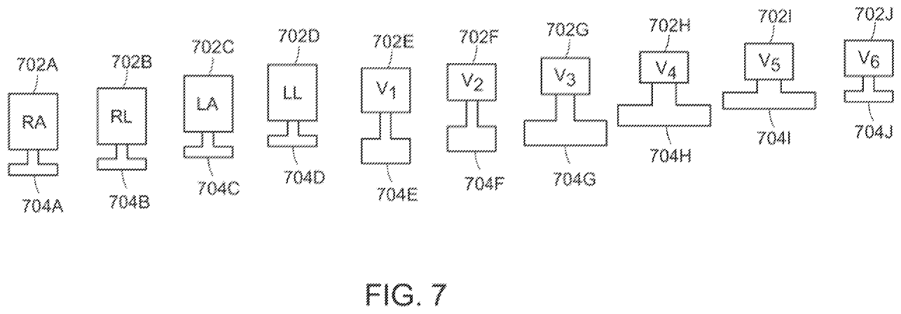

FIG. 7 is a schematic diagram of wireless contact leads RA, RL, LA, LL, and V1-V6, in accordance with some embodiments. Top portions 702A-702J for contact leads RA. RL, LA. LL, and V1-V6 may be appropriately labeled to identify the contact leads. This way a doctor and/or technician could quickly identify the correct contact lead without confusion. In some embodiments, top portions 702A-702J may be assigned different colors or shades, that can be a hue or mixture of pure colors, corresponding to a contact lead RA, RL, LA, LL, and V1-V6. In some embodiments, top portions 702A-702J may be assigned the same colors or shading of one or more colors as receivable slots 602A-602J. In some embodiments, top portions 702A-702J may have different shapes and sizes.

Moreover, each contact lead RA, RL, LA, LL, and V1-V6 may include a base 704A-704J, respectively, having different sizes relative to each other. This way a doctor and/or technician would not place a contact lead in a wrong receivable slot for charging. In some embodiments, bases 704A-704J may have different shapes and sizes. In some embodiments, bases 704A-704J may have different shapes and sizes. In some embodiment, the top portion may include different shapes and/or colors to identify a wireless contact lead from the plurality of contact leads.

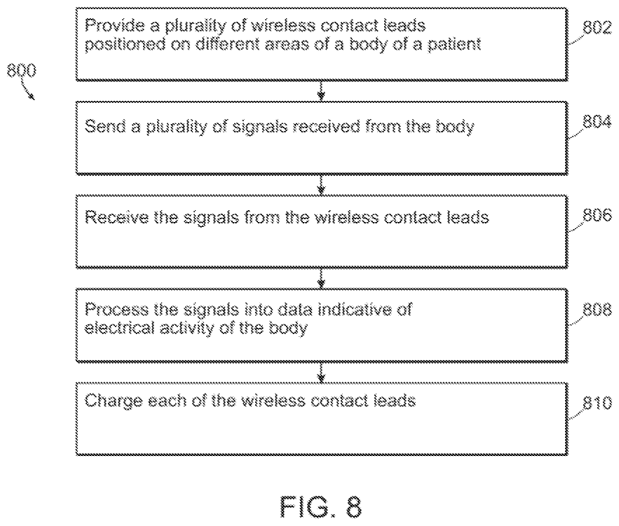

FIG. 8 is a process flowgraph of operations performed by a wireless ECG system, in accordance with some embodiments. Process 800 includes providing a plurality of wireless contact leads (such as wireless contact leads RA, RL, LA, LL, and V1-V6) positioned on different areas of a body (such as body 100 shown in other figures) of a patient (Step 802). Process 800 includes sending, by each of the wireless contact leads, wirelessly a plurality of signals received from a heart of the patient (Step 804). The signals are received from the wireless contact leads using processing unit (such as ECG module 202 or computer system) (Step 806). Process 800 includes processing, using the processing unit, the signals into data indicative of electrical activity of the body (Step 808). Furthermore, process 800 includes charging, using a charging component (such as charging component 400), each of the wireless contact leads (Step 810). The charging component includes a plurality of slots (such as receivable slots 602A-602J) for charging each of the wireless contact leads.

Reference in the specification to “one implementation” or “an implementation” means that a particular feature, structure, or characteristic described in connection with the implementation is included in at least one implementation of the disclosure. The appearances of the phrase “in one implementation,” “in some implementations,” “in one instance,” “in some instances,” “in one case,” “in some cases,” “in one embodiment,” or “in some embodiments” in various places in the specification are not necessarily all referring to the same implementation or embodiment.

Finally, the above descriptions of the implementations of the present disclosure have been presented for the purposes of illustration and description. It is not intended to be exhaustive or to limit the present disclosure to the precise form disclosed. Many modifications and variations are possible in light of the above teaching. It is intended that the scope of the present disclosure be limited not by this detailed description, but rather by the claims of this application. As will be understood by those familiar with the art, the present disclosure may be embodied in other specific forms without departing from the spirit or essential characteristics thereof. Accordingly, the present disclosure is intended to be illustrative, but not limiting, of the scope of the present disclosure, which is set forth in the following claims.

What is claimed is:

1. A wireless electrocardiogram (ECG) system, comprising:
a plurality of wireless contact leads for positioning on different areas of a body of a patient, each of the wireless contact leads wirelessly sends a plurality of signals received from the body of the patient:
an ECG module for receiving the signals from the wireless contact leads, and processing the signals into data indicative of electrical activity of the body; and
a charging component coupled to the ECG module, wherein the charging component includes a plurality of slots for charging each of the wireless contact leads.

2. The wireless ECG system of claim 1, wherein the wireless contact leads utilize a wireless communication standard to send the signals to the ECG module.

3. The wireless ECG system of claim 1, wherein the ECG module comprises a communication module for managing communications between the wireless contact leads and the ECG module.

4. The wireless ECG system of claim 1, wherein the ECG module is coupled to a computer system for displaying the data.

5. The wireless ECG system of claim 1, wherein each of the wireless contact leads comprises a top portion, middle portion, and base.

6. The wireless ECG system of claim 5, wherein the top portion comprises markings to identify a wireless contact lead from the plurality of contact leads.

7. The wireless ECG system of claim 5, wherein the top portion comprises different colors and/or shades to identify a wireless contact lead from the plurality of contact leads.

8. The wireless ECG system of claim 5, wherein the top portion comprises different shapes and/or colors to identify a wireless contact lead from the plurality of contact leads.

9. The wireless ECG system of claim 5, wherein the base is positioned in a receivable slot for charging a wireless contact lead.

10. The wireless ECG system of claim 9, wherein the base comprises different shapes and/or colors to identify a receivable slot from the plurality of receivable slots for charging the wireless contact lead.

11. A charging system comprising:
a plurality of wireless contact leads configured to receive signals from a body to perform an electrocardiogram (ECG); and
a charging component configured to have a plurality of receivable slots for charging each of the wireless contact leads, each of the receivable slots assigned to a particular wireless contact lead from the plurality of wireless contact leads, wherein the charging component comprises a plurality of indicators to indicate the status of each of the wireless contact leads positioned in their respective receivable slots.

12. A method for performing operations of a wireless electrocardiogram (ECG) system, the method comprising:
providing a plurality of wireless contact leads positioned on different areas of a body of a patient,
sending, by each of the wireless contact leads, a plurality of signals received from the body of the patient;
receiving, using a processing unit, the signals from the wireless contact leads;
processing, using the processing unit, the signals into data indicative of electrical activity of the body; and
charging, using a charging component, each of the wireless contact leads, wherein the charging component includes a plurality of slots for charging each of the wireless contact leads.

13. The method of claim 12, wherein sending the plurality of signals comprises utilizing a wireless communication standard to send the signals to the processing unit.

14. The method of claim 12, wherein the processing unit comprises a communication module for managing communications between the wireless contact leads and the processing unit.

15. The method of claim 12, wherein processing the signals comprises coupling the processing unit to a computer system for displaying the data.

16. The method of claim 12, wherein each of the wireless contact leads comprises a top portion, middle portion, and base.

17. The method of claim 16, wherein the top portion comprises markings to identify a wireless contact lead from the plurality of contact leads.

18. The method of claim 16, wherein the top portion comprises different colors and/or shades to identify a wireless contact lead from the plurality of contact leads.

19. The method of claim 16, wherein the top portion comprises different shapes or sizes to identify a wireless contact lead from the plurality of contact leads.

20. The method of claim 16, wherein charging each of the wireless contact leads comprises positioning the base in a receivable slot for charging a wireless contact lead.

21. The method of claim 20, wherein the base comprises different shapes and/or colors to identify a receivable slot from the plurality of receivable slots for charging the wireless contact lead.

22. An electrocardiogram (ECG) system comprising:
a plurality of wireless contact leads; and
a processing unit configured to receive signals from the wireless contact leads and process the signals into data indicative of electrical activity of a body of a user.

23. The ECG system of claim 22, wherein the wireless contact leads utilize a wireless communication standard to send the signals to the processing unit.

24. The ECG system of claim 22, wherein the processing unit comprises a communication module for managing communications between the wireless contact leads and the processing unit.

25. The ECG system of claim 22, wherein the processing unit displays the data for viewing.

26. The ECG system of claim 22, wherein each of the wireless contact leads comprises a top portion, middle portion, and base.

27. The ECG system of claim 26, wherein the top portion comprises markings to identify a wireless contact lead from the plurality of contact leads.

28. The ECG system of claim 26, wherein the top portion comprises different colors and/or shades to identify a wireless contact lead from the plurality of contact leads.

29. The ECG system of claim 26, wherein the top portion comprises different shapes and/or colors to identify a wireless contact lead from the plurality of contact leads.

30. The ECG system of claim 26, wherein the base is positioned in a receivable slot for charging a wireless contact lead.

31. The ECG system of claim 30, wherein the base comprises different shapes and/or colors to identify a receivable slot from the plurality of receivable slots for charging the wireless contact lead.

32. A wireless contact lead, comprising:
a top portion configured to have markings indicative of a location of the wireless contact lead on a body; and
a base coupled to the top portion configured to be positioned in a receivable slot of a charging system for charging the wireless contact lead.

33. The wireless contact lead of claim 32, wherein the markings are configured to visually identify a wireless contact lead from a plurality of contact leads.

34. The wireless contact lead of claim 32, wherein the markings are configured to have different colors and/or shades to identify a wireless contact lead from the plurality of contact leads.

35. The wireless contact lead of claim 32, wherein the top portion is configured to have different shapes and/or colors to identify a wireless contact lead from the plurality of contact leads.

36. The wireless contact lead of claim 32, wherein the base is configured to have different shapes and/or colors to identify a receivable slot from the plurality of receivable slots for charging the wireless contact lead.
